(19) United States
(12) Patent Application Publication
(10) Pub. No.: US 2010/0010298 A1
(43) Pub. Date: Jan. 14, 2010

(54) ENDOSCOPIC TRANSLUMENAL FLEXIBLE OVERTUBE

(75) Inventors: Gregory J. Bakos, Mason, OH (US); William D. Fox, New Richmond, OH (US); James T. Spivey, Cincinnati, OH (US); Bernard C. McDermott, Kiltimagh (IE); Micheal E. Kelly, Tubber (IE)

Correspondence Address: K&L GATES LLP, 535 SMITHFIELD STREET, PITTSBURGH, PA, 15222, US

(73) Assignee: Ethicon Endo-Surgery, Inc., Cincinnati, OH (US)

(21) Appl. No.: 12/172,752

(22) Filed: Jul. 14, 2008

(51) Int. Cl. A61B 1/01; A61B 1/015

(52) U.S. Cl. 600/106; 600/114; 600/115

(57) ABSTRACT

Apparatus, system, and method for use with an endoscope are disclosed. A flexible overtube having a proximal end and a distal end defines a hollow lumen therebetween to receive a flexible shaft portion of an endoscope therein. The proximal end of the flexible overtube is configured to remain outside of a patient and the distal end is configured to enter the patient through a natural orifice. At least one fluid tight seal is located at the proximal end of the flexible overtube to prevent leakage of fluids around the flexible shaft of the endoscope when the flexible shaft of the endoscope is positioned within the flexible overtube. The system further includes a flexible endoscope. The method includes introducing the system into a patient though a natural orifice of the patient and performing an endoscopic translumenal procedure.

### BACKGROUND

In laparoscopic surgical procedures, a small incision is made in the body. A trocar is inserted through the incision. The trocar receives an elongate shaft of a surgical device to position a distal end of the shaft at a surgical worksite. In some endoscopic procedures, the elongate shaft of the surgical device is inserted through a natural orifice of the patient, such as the mouth, vagina, or anus, and is advanced along an internal pathway to position the distal end of the device at the surgical worksite. Endoscopic procedures typically require the use of a flexible shaft to accommodate the tortuous pathway of the body lumen, whereas rigid shafts can be used in laparoscopic procedures. These tools can be used to engage and/or treat tissue in a number of ways to achieve a diagnostic or therapeutic effect.

Endoscopic surgery can be used to access the abdominal cavity via natural openings (mouth, anus, vagina, urethra) of the body and through the peritoneal lining of the abdominal cavity. The size and shape of instruments that may be passed through a body lumen to perform a medical procedure in the abdominal cavity are greatly restricted due to the anatomical properties of the lumen. General surgeons, gastroenterologists, and other medical specialists, routinely use flexible endoscopes for intralumenal (within the lumen of the alimentary canal) examination and treatment of the upper gastrointestinal (GI) tract, via the mouth, and the lower GI tract, via the anus. In these procedures, the physician pushes the flexible endoscopes into the lumen, periodically pausing to articulate the distal end of the endoscope. In this manner, the physician may navigate the crooked passageway of the upper GI past the pharynx, through the esophagus and gastroesophageal junction, and into the stomach. In the process, the physician must take great care not to injure the delicate mucosal lining of the lumen, which has a non-circular cross sectional configuration when relaxed, but can stretch open to a diameter in the range of about 15-25 mm during the insertion procedure.

During translumenal procedures, a puncture must be formed in the stomach wall, gastrointestinal tract, or other epithelialized natural orifice to access the peritoneal cavity. A needle knife is one device often used to form such a puncture. The needle knife is inserted through the working channel of the endoscope and utilizes energy to penetrate through the tissue. A guidewire is then feed through the endoscope and is passed through the puncture in the stomach wall and into the peritoneal cavity. When the needle knife is removed, the guidewire is left as a placeholder. A balloon catheter is then passed over the guidewire through the working channel of the endoscope to position the balloon within the opening in the stomach wall. The balloon is inflated to increase the size of the opening, thereby enabling the endoscope to push against the rear of the balloon and to be feed through the dilated opening and into the peritoneal cavity. Once the endoscope is positioned within the peritoneal cavity, numerous procedures can be performed with instruments introduced through the one or more working channels of the endoscope.

While current methods and devices used to insert endoscopes into a natural orifice of a patient are effective, one drawback is that there is no sealed conduit for the endoscope to pass in and out of the peritoneal cavity multiple times while maintaining the access location and maintaining the peritoneal cavity insufflated. Traditional overtubes that slide over the endoscope generally are not sealed and thus limits their use to application where the peritoneal cavity is not insufflated.

Accordingly, there remains a need for improved endoscopic translumenal methods and devices.

### BRIEF DESCRIPTION OF THE DRAWINGS

The various embodiments will be more fully understood from the following detailed description taken in conjunction with the accompanying drawings, in which:

FIG. 1 is a side view of one embodiment of a flexible endoscopic translumenal overtube assembly comprising a flexible endoscope disposed within one embodiment of a flexible overtube.

FIG. 2 is a side view of one embodiment the flexible overtube of the assembly shown in FIG. 1.

FIG. 3 is a side view of one embodiment of the endoscope of the assembly shown in FIG. 1.

FIG. 4 is cross-sectional view of one embodiment of the flexible sheath portion of the flexible overtube taken along section line 1-4 as shown in FIG. 2.

FIG. 5 is a partial cut-away view of one embodiment of the flexible sheath to show a method of fabricating the flexible sheath.

FIG. 6 is a distal end view of one embodiment of the flexible overtube shown in FIG. 2.

FIG. 7A is a cross-sectional view of one embodiment of the endoscopic end cap.

FIG. 7B is a distal end view of one embodiment of the endoscopic end cap.

FIG. 8A illustrates one embodiment of the endoscopic end cap slidably inserted over the outside diameter of the distal end of the flexible endoscopic shaft.

FIG. 8B illustrates the distal end of the flexible endoscopic shaft introduced into the distal end of one embodiment of the flexible overtube through the hollow lumen of the flexible overtube.

FIG. 9 illustrates one embodiment of a modular endoscopic overtube.

FIGS. 10A-N illustrate one embodiment of a method of introducing an endoscopic translumenal surgical device through the wall of a hollow organ during an endoscopic translumenal surgical procedure, where:

FIG. 10A illustrates one embodiment of a flexible endoscopic shaft of an endoscope inserted inside a stomach wall and a distal end of the endoscopic end cap positioned in contact with an internal portion of the stomach wall.

FIG. 10B illustrates an isolated tissue wall suctioned into one embodiment of an end cap and a flexible hollow tubular stylette advanced over a solid central needle.

FIG. 10C illustrates one embodiment of a solid central needle advanced to pierce or puncture an isolated stomach wall tissue.

FIG. 10D illustrates one embodiment of the tubular stylette extended or advanced when the isolated stomach wall tissue is punctured with the solid central needle.

FIG. 10E illustrates one embodiment of a solid central needle comprising a sharp distal end and a dilating portion to pierce and spread the isolated stomach wall tissue to minimize cutting vessels and tissue.

FIG. 10F illustrates one embodiment of a tubular stylette and a deflated balloon advanced through a puncture site of the isolated tissue, wherein the balloon is positioned simultaneously in the isolated stomach wall tissue and partially inside the distal end of the flexible overtube.

FIG. 10G illustrate one embodiment of an insufflated balloon to dilate the puncture in the isolated stomach wall tissue.

FIG. 10H illustrates one embodiment of a flexible endoscopic translumenal overtube assembly comprising a flexible overtube and an endoscope with an endoscopic end cap advanced through the dilated opening formed in the stomach wall tissue.

FIG. 10I illustrates one embodiment of a spring for biasing a tubular stylette introduced over a solid central needle.

FIG. 11A is a side view of one embodiment of a steerable segment of one embodiment of the flexible overtube shown in FIGS. 1 and 2.

FIG. 11B is a top view of one embodiment of the steerable segment shown in FIG. 11A.

FIG. 11C is a bottom view of one embodiment of the steerable segment shown in FIG. 11A.

FIG. 12 is a side view of one embodiment of the steerable segment shown in FIGS. 11A-C.

FIG. 13 is a perspective view of a portion of the steerable segment shown in FIGS. 11A-C and 12.

FIG. 13A is a cross-sectional view of a wall portion of the steerable segment shown in FIG. 13.

FIG. 13B is a cross-sectional view of a wall portion of the steerable segment shown in FIG. 13.

FIG. 14 is a cross-sectional view of the steerable segment and a middle segment of the flexible overtube shown in FIGS. 1 and 2.

FIG. 15 illustrates a steerable segment of the flexible endoscopic translumenal overtube assembly shown in FIG. 1 in an actuated state.

FIG. 16 is a side view of one embodiment of a steerable tube comprising an interrupted spiral cut pattern of slits.

FIG. 17 is a side view of one embodiment of a steerable tube comprising a spiral cut pattern slits.

FIG. 18 illustrates one embodiment of a steerable segment comprising a multi-lumen steerable tube and a flexible segment.

### DESCRIPTION

Methods and devices are provided for a flexible endoscopic translumenal overtube for receiving a flexible endoscope therethrough. Certain embodiments of a flexible endoscopic translumenal overtube will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting example embodiments and that the scope of the embodiments described in this application is defined solely by the claims. The features illustrated or described in connection with one embodiment may be combined with the features of other embodiments. Such modifications and variations are intended to be included within the scope of this application.

It will be appreciated that the terms “proximal” and “distal” are used herein with reference to a clinician manipulating one end of an instrument that protrudes out of a natural orifice (or opening) of the patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the drawings. However, surgical instruments may be used in many orientations and positions, and these terms are not intended to be limiting and absolute.

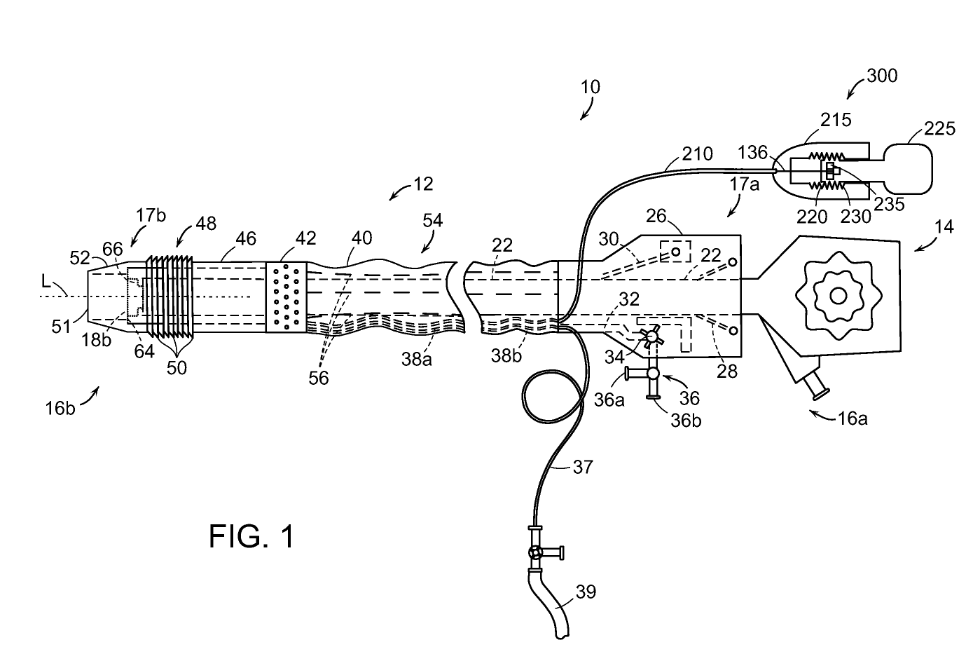

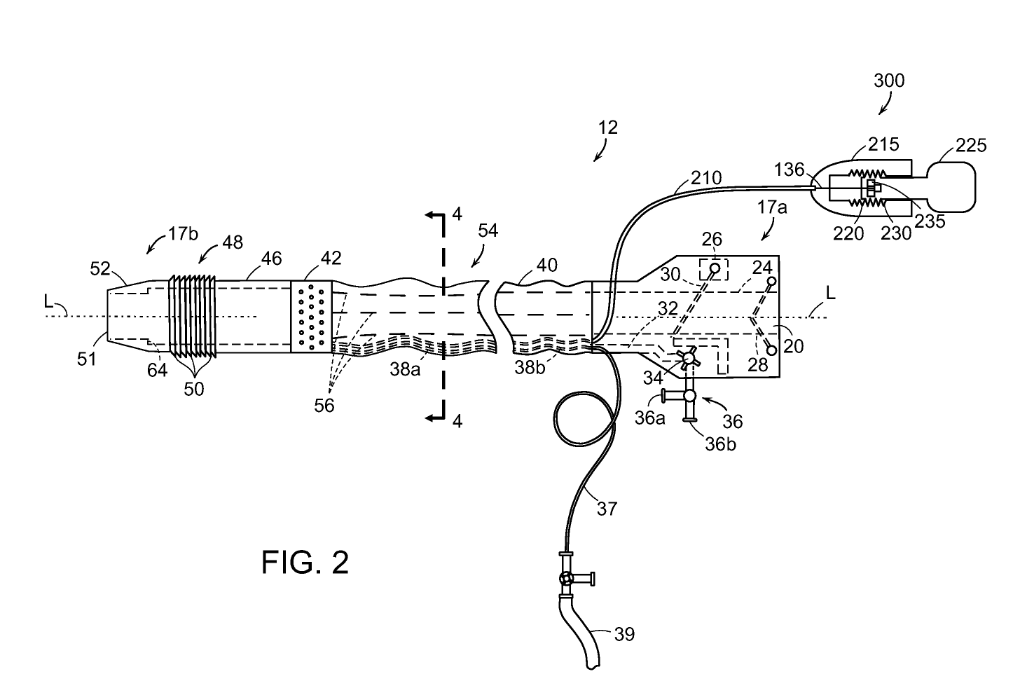

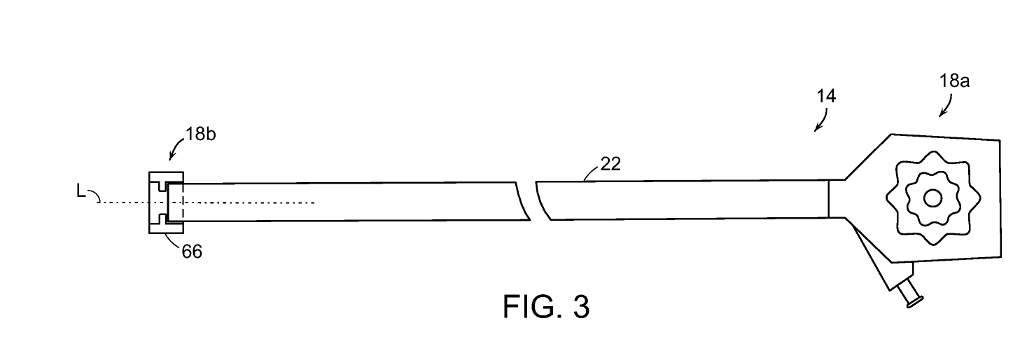

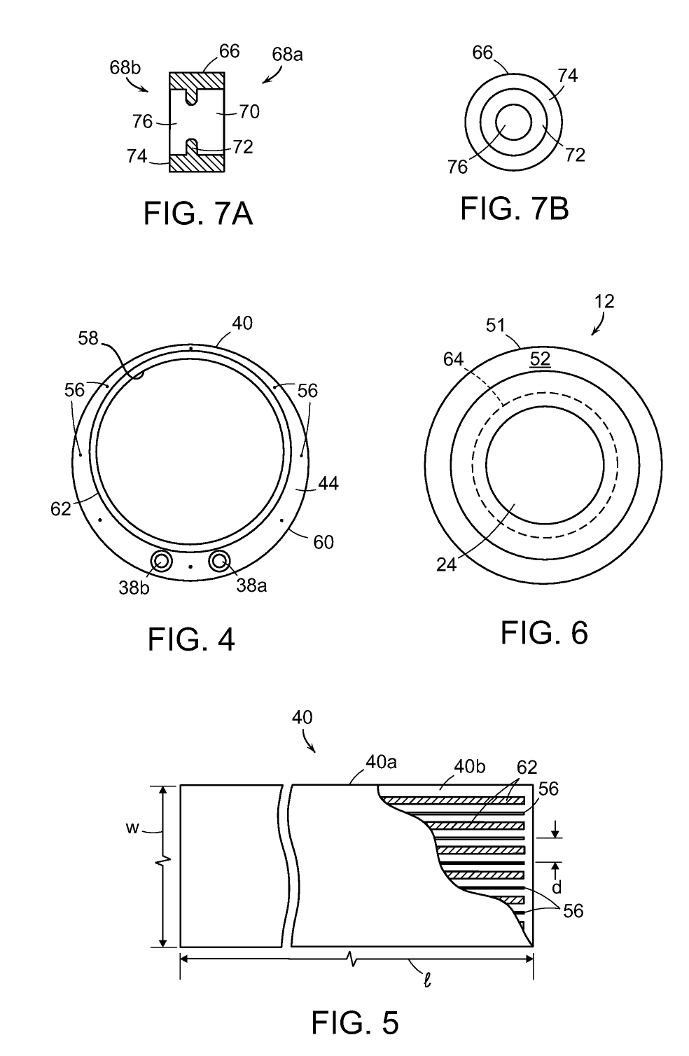

FIG. 1 is a side view of one embodiment of a flexible endoscopic translumenal overtube assembly 10 comprising a flexible endoscope 14 disposed within a flexible overtube 12. The flexible endoscopic translumenal overtube assembly 10 extends substantially along a longitudinal axis “L.” FIG. 2 is a side view of the flexible overtube 12 of the assembly 10 shown in FIG. 1. The flexible overtube 12 is coupled to a steerable segment 46 that is coupled to an actuation handle 300 for actively articulating the steerable segment 46 away from a neutral axis, e.g., the longitudinal axis “L,” in a radius of curvature defined by a pattern or series of cuts formed on a steerable element of the steerable segment 46. FIG. 3 is a side view of the endoscope 14 portion of the assembly 10 shown in FIG. 1. With reference to FIGS. 1-3, in one embodiment, the endoscopic translumenal overtube assembly 10 comprises a proximal end 16a and a distal end 16b. The proximal end 16a remains out of the patient and the distal end 16b is inserted through a natural orifice, such as the mouth, vagina, or anus, and is advanced along a pathway to position a distal end of the device at a surgical site. The flexible overtube 12 comprises a flexible hollow body having a proximal end 17a and a distal end 17b and defining an opening 24 extending therebetween. The endoscope 14 also comprises a proximal end 18a and a distal end 18b. The distal end 18b of the endoscope 14 is slidably introduced into an opening 20 defined at the proximal end 16a of the flexible overtube 12. The distal end 18b of the endoscope 14 is inserted through the proximal opening 20 of the flexible overtube 12. The distal end 18b and a flexible shaft 22 portion of the endoscope 14 are advanced through the opening 24 defined by the flexible overtube 12 until the distal end 18b of the endoscope 14 engages the distal end 17b of the flexible overtube 12, as discussed in more detail below. A middle segment 54 of the flexible overtube 12 comprises a flexible sheath 40 defining an opening along the longitudinal axis “L” suitably sized to receive the flexible shaft 22 of the endoscope 14 with some clearance for insufflation. The flexible sheath 40 is formed into a longitudinally extending tube such that the outside diameter 60 (FIG. 4) of the flexible sheath 40 can be minimized to a suitable dimension required to pass through a desired anatomical lumen or body cavity. The flexible shaft 22 of the endoscope 14 can be moved independently of the flexible sheath 40. The flexible sheath 40 can be left in place in the anatomical lumen as a conduit for reintroducing therein the flexible shaft 22 of the endoscope 14 or for introducing therein other instruments for use within the anatomical lumen or body cavity. The endoscope 14 comprises one or more working channels to introducing various surgical instruments to the surgical worksite within the patient.

With reference to FIGS. 1 and 2, in one embodiment the proximal end 17a of the flexible overtube 12 comprises a seal system 26 to provide a fluid tight seal regardless of whether the endoscope 14 is located within the flexible overtube 12. It will be appreciated that a fluid tight seal refers to a seal sufficient to maintain pneumoperitoneum fluid pressure with incidental gaseous or fluid leakage. In one embodiment, at least one fluid tight seal 28 is provided at the proximal end 17a of the flexible overtube 12. The at least one fluid tight seal 28 prevents leakage of fluids around the flexible shaft 22 portion of the endoscope 14 positioned within the flexible overtube 12. In one embodiment, an additional fluid tight seal 30 may be provided near the proximal end 17a of the flexible overtube 12 to prevent leakage of fluid through the inside of the flexible overtube 12 when the opening 24 is free of devices, such as the endoscope 14. The first and second seals 28, 30 may have a variety of configurations. In various embodiments, however, the first and second seals 28, 30 may be configured to provide fluid tight seals around an endoscope having a size range between about 5 mm to about 13 mm. In other embodiments, the first and second seals 28, 30 may be configured to provide suitable fluid tight seals around endoscopes having other sizes. Therefore, the embodiments should not be limited in this context.

In one embodiment, the proximal end 17a of the flexible overtube 12 comprises an opening 32 distal to the first and second seals 28, 30. The opening 32 can be selectively opened and closed to allow passage of fluids from inside the flexible overtube 12 to the outside of the flexible overtube 12. The opening 32 is fluidically coupled to a valve 34 to enable the opening to fluidically couple to a fluid connection 36. The valve 34 may have a variety of configurations, and in the illustrated embodiment is a stopcock type valve. The fluid connection 36 is configured with one or more fluid ports 36a, 36b to fluidically couple either a positive pressure source (e.g., insufflation source) or a negative pressure source (e.g., suction source) to the flexible overtube 12. The first fluid port 36a is fluidically coupled to the interior of the flexible sheath 40 portion of the flexible overtube 12. The first fluid port 36a also may be fluidically coupled to an insufflation system suitable for insufflating and maintaining pneumoperitoneum fluid pressure within the peritoneal cavity during a surgical or diagnostic procedure. The first and second seals 28, 30 provide fluid tight seals to maintain the pneumoperitoneum fluid pressure to prevent the peritoneal cavity from deflating during the procedure. The first fluid port 36a may be a luer connection to couple to a syringe or insufflator. In one embodiment, the first fluid port 36a may be a female luer connection.

First and second lumens 38a, 38b are embedded within the flexible sheath 40 portion of the flexible overtube 12 and are fluidically separated from each other and from the interior of flexible sheath 40. In one embodiment, the first lumen 38a forms a conduit from the proximal end of the flexible overtube 12 to the steerable segment 46. In one embodiment, the first lumen 38a is sized to receive a pull cable 136 suitable for actuating the steerable segment 46. The pull cable 136 may be contained within a coil pipe assembly 210 used in the actuation of the steerable segment 46, as described with particularity below. In one embodiment, the second lumen 38b is fluidically coupled to a suction collar 42, which is in fluid communication with the exterior surface of the flexible sheath 40. The proximal end of the second lumen 38b is fluidically coupled to a suction source via flexible tubing 37. The flexible tubing 37 may be coupled to an endoscope, syringe, or positive or negative pressure source via a flexible tubing 39. The suction collar 42 can be used to evacuate the inside of an organ while the distal end 17b of flexible overtube 12 is positioned through the wall of the organ. This may be particularly useful in procedures where the distal end 17b of the flexible overtube 12 is positioned in the stomach, which may balloon to a size that may hinder the procedure. With the flexible tubing 39 coupled to a negative pressure source, the clinician may deflate the organ through the suction collar 42 without repositioning the flexible overtube 12.

The distal end 17b of the flexible overtube 12 may comprise a tapered segment 52, which provides a smooth transition while passing through an internal lumen or a dilated orifice formed in the tissue wall of an organ. A tissue gripping stability feature 48 may be formed near the distal end 17b of the flexible overtube 12 on an exterior surface thereof. The stability feature 48 helps position the distal end 17b of the flexible overtube 12 in the patient's body, e.g., the penetrated tissue wall of an organ. The stability feature 48 is configured to allow the distal end 17b of the flexible overtube 12 to easily pass through a dilated orifice formed through the tissue wall of an organ and provides tissue gripping features to prevent the distal end 17b from being easily pulled back through the dilated orifice in the tissue wall of the organ. The stability feature 48 may have a variety of configurations. In the illustrated embodiment, the stability feature 48 comprises a plurality of annular rings 50 formed on the outer surface of the flexible overtube 12. In the illustrated embodiment, the annular rings 50 have a triangular cross section. In other embodiments, the stability feature 48 may comprise a balloon disposed on the outer surface of the flexible overtube 12 that allows easy passage through a tissue wall when deflated and maintains the position of the distal end 17b of the flexible overtube 12 when inflated.

The actuation handle 300 is used to apply tension to the pull cable 136 to bend the steerable segment 46. The actuation handle 300 comprises a housing 215 including internal threads 220 on its interior surface, and a knob 225 containing mating threads 230 on its external surface. The pull cable 136 is fixed to the knob 225 through a rotational coupling 235 and the coil pipe 210 is fixed to the housing 215. Clockwise rotation of the knob 225 results in translation of the knob 225 relative to the housing 215 and applies tension to the pull cable 136. Rotation is continued until the tension in the pull cable 136 creates the desired amount of angulation off of the neutral axis “L” of the steerable segment 46. The actuation handle 300 may then be placed aside during a portion of the procedure with the steerable segment 46 remaining in its flexed state. When desired, the knob 225 can then be rotated counterclockwise to reduce tension in the pull cable 136, allowing the steerable segment 46 to return to a straight position.

FIG. 4 is cross-sectional view of the flexible sheath 40 portion of the flexible overtube 12 taken along section line 4-4 as shown in FIG. 2. The fluid tight first and second lumens 38a,b are embedded within a wall 44 portion of the flexile sheath 40 and extend along the length of the flexible overtube 12. The embedded lumens 38a,b may have a variety of configurations. In one embodiment, the first and second lumens 38a,b may be made of coil pipes for flexibility and may be coated with a polyethylene (PET) coating. In one embodiment, the inner diameter of each of the embedded lumens 38a,b may be about 1 mm. In one embodiment, a distal end of the second lumen 38b is fluidically coupled to the suction collar 42. Accordingly, the suction collar 42 may be used to draw fluid from the inside of a patient's body and into the fluid tight second lumen 38b when the proximal end of the second lumen 38b is connected to a negative pressure source via the flexible tubing 39. In other embodiments, the first and second lumen 38a,b each may be configured to receive the pull cable 136 within the elongate hollow portion of the embedded lumen.

With reference now to FIGS. 1-4, in one embodiment the middle segment 54 of the flexible overtube 12 is located between the proximal and distal ends 17a,b. The middle segment 54 may have a variety of configurations. In the illustrated embodiment the middle segment 54 comprises the flexible sheath 40 sized to fit comfortably over the flexible shaft 22 of the endoscope 14. In one embodiment, the flexible sheath 40 may be formed of any suitable sheath material having a minimal wall thickness but with sufficient strength and toughness to resist tears and punctures when introduced over the flexible shaft 22. The sheath material also should be leak proof, biocompatible, lubricious (e.g., slippery, low friction), and should provide a fluid tight barrier between the flexible shaft 22 of the endoscope 14 and the internal body lumen in which the flexible overtube is inserted. In one embodiment, the flexible sheath 40 may be formed of TIVEK®. Those skilled in the art will appreciate that TYVEK® material can be configured to form a fluid tight barrier, is highly rip resistant, biocompatible, and is naturally lubricious.

In one embodiment, the flexible sheath 40 may comprise longitudinally disposed reinforcing structural members 56 disposed along the length of the flexible sheath 40. The structural members 56 provide columnar strength to the flexile sheath 40 to assist in the independent movement of the flexible shaft 22 of the endoscope 14 relative to the flexible sheath 40. Although the reinforcing structural members 56 can have a variety of configurations, in one embodiment, the reinforcing structural members 56 in the illustrated embodiment are configured as longitudinally extending spaced apart elongate wires.

FIG. 5 is a partial cut-away view of one embodiment of the flexible sheath 40 to show a method of fabricating the flexible sheath 40. As illustrated, the flexible sheath 40 comprises a first layer 40a and a second layer 40b. The first and second layers 40a,b have a suitable length “l” and width “w” to accommodate the final configuration of the flexible sheath 40. For example, the first and second layers may have a width “w” of about 8 cm to accommodate a range of flexible endoscopic shafts 22. The length “l” is variable and in one embodiment may be about 100 cm. These dimensions are not limited and may be varied to accommodate any desired length “l” and width “w.” A plurality of longitudinally extending structural members 56 are disposed between the first and the second sheaths 40a,b and are separated by a distance “d.” In one embodiment, the distance between the structural members 56 is about 13 mm. A bonding element 62 may be disposed in the spaces between the spaced apart structural members 56 such that the structural members 56 and the bonding elements 62 are alternately positioned along the width “w” of the first and second layers 40a,b. The first and second layers 40a,b with the structural members 56 and the bonding elements 62 disposed therebetween are bonded by the bonding elements 62 to form a unitary structure that can be rolled into a tubular shape to form the flexible sheath 40. As previously discussed, the flexible sheath 40 defines the opening 24 for receiving therein a suitably sized flexible endoscopic shaft 22. The flexible sheath 40 may have various thicknesses and in one embodiment may have a thickness of about 0.5 mm (e.g., about 20 mils). Although the various components of the flexible sheath 40 may have many configurations, in the illustrated embodiment, the first and second layers 40a,b can be made of TYVEK® sheets and the structural members 56 can be made of NITINOL® wire having a diameter of about 0.0335 mm, for example. In other embodiments, the reinforcing structural members 56 may be configured steel springs or polymeric columns. In one embodiment, a structural reinforcing structural member 56 may be configured as an external endorail longitudinally extending along an exterior surface of the flexible sheath 40. In one embodiment, the bonding elements 62 may be formed of two-part epoxy.

FIG. 6 is a distal end view of one embodiment of the flexible overtube 12. Referring to FIGS. 1-3 and 6, in the illustrated embodiment, the distal end 17b of the flexible overtube 12 comprises a generally cylindrical end cap 51 with the tapered surface 52 and an internal circumferential radial protruding wall 64 that is configured to engage an endoscopic end cap 66 suitable to fit over the distal end 18b of the flexible endoscopic shaft 22. The wall 64 is dimensioned to stop the distal end 18b flexible endoscopic shaft 22 with the endoscopic end cap 66 from protruding through the distal end 17b of the flexible overtube 12. Although the cylindrical end cap 51 can have a variety of configurations, in one embodiment the cylindrical end cap 51 may be formed of molded soft plastic material, for example.

FIG. 7A is a cross-sectional view of the endoscopic end cap 66 and FIG. 7B is a distal end view the endoscopic end cap 66. The endoscopic end cap 66 comprises a proximal end 68a and a distal end 68b. The proximal end 68a defines an opening 70 configured to slidably receive the distal end 18b of the flexible endoscopic shaft 22. The distal end 18b of the flexible endoscopic shaft 22 butts against and engages a circumferential radial projection 72 to prevent the distal end 18b of the flexible endoscopic shaft 22 from protruding through the endoscopic end cap 66. The distal end 68b defines an opening 76 for receiving therethrough the distal end 18b of the flexible endoscopic shaft 22 when the end cap 66 is removed therefrom. The distal end 68b comprises a circumferential portion 74 configured to engage the circumferential radial protruding wall 64 of the end cap 51 of the flexible overtube 12. Although the endoscopic end cap 66 can have a variety of configurations, in one embodiment the endoscopic end cap 66 may be formed of plastic, such as clear see-through polycarbonate material, for example.

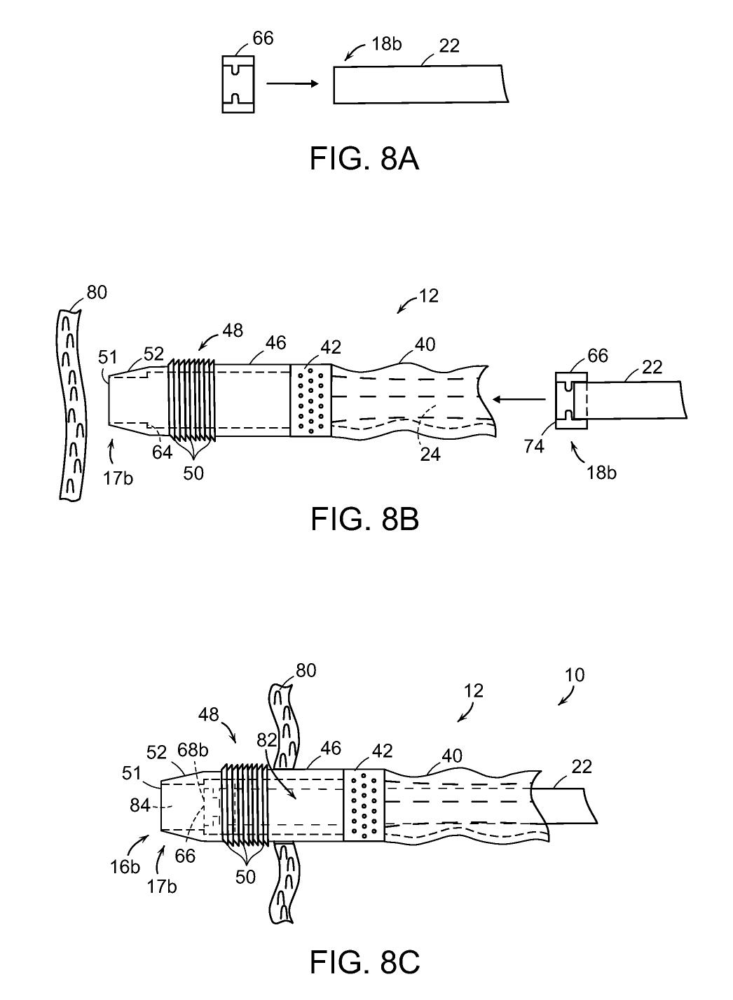

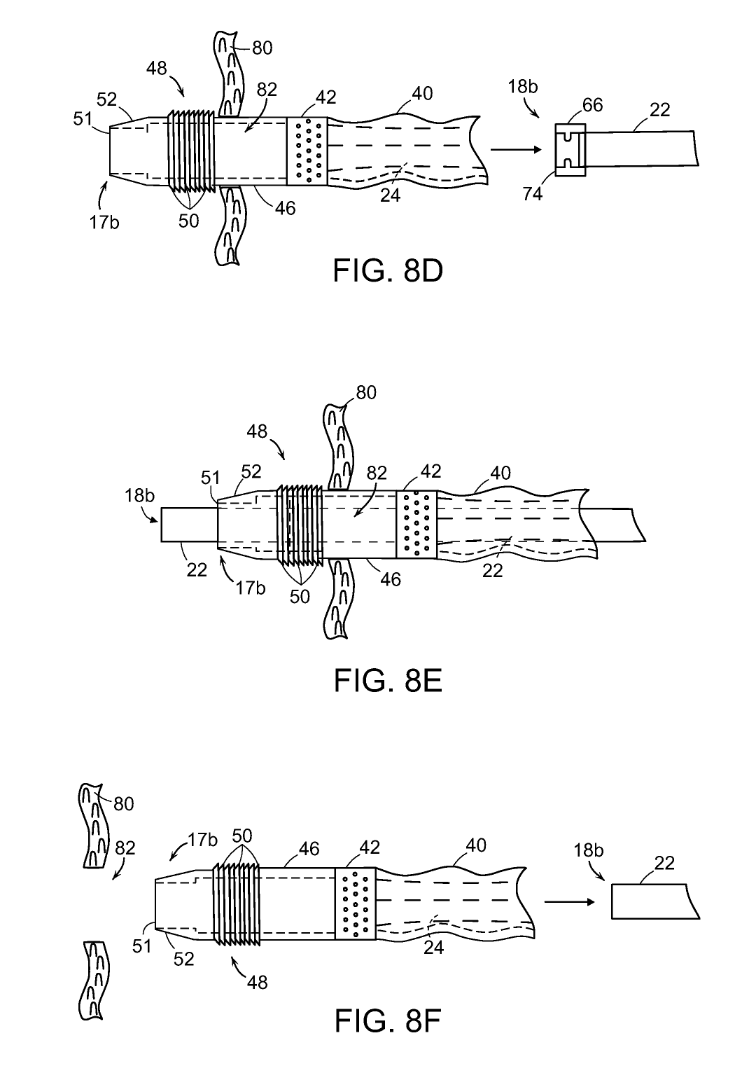

A sequence of steps for using the flexible endoscopic translumenal overtube assembly 10 is illustrated in FIGS. 8A-F. Initially, the flexible overtube 12 is inserted into a natural orifice of the patient that is suitable to reach the tissue treatment region. As shown in FIG. 8A, the endoscopic end cap 66 is slidably inserted over the outside diameter of the distal end 18b of the flexible endoscopic shaft 22.

As shown in FIG. 8B, the distal end 18b of the flexible endoscopic shaft 22 is introduced into the distal end 17b of the flexible overtube 12 through the opening 24 of the flexible overtube 12. The flexible endoscopic shaft 22 is inserted into the cylindrical end cap 51 of the flexible overtube 12 until the circumferential portion 74 of the endoscopic end cap 66 engages the circumferential radial protruding wall 64 of the end cap 51 of the flexible overtube 12. The end caps 51, 66 fit together. The flexible endoscopic translumenal overtube assembly 10 is then located in proximity to a tissue wall 80.

As shown in FIG. 8C, the distal tip 16b of the flexible endoscopic translumenal overtube assembly 10 is inserted through a dilated orifice 82 formed in the tissue wall 80. An example of how to puncture the tissue wall 80 and dilate the resulting orifice is discussed in more detail below. A space 84 is provided between the distal end 68b of the endoscopic end cap 66 and the distal end 17b of the flexible overtube 12. The space 84 is suitable to enable a dilation balloon to be inflated therein. Once the distal end 17b of the flexible overtube 12 is pushed through the dilated orifice 82 in the tissue wall 80, the tissue gripping stability feature 48 grips the tissue wall 80 to prevent the distal end 17b of the flexible overtube 12 from pulling away from the dilated opening 80.

As shown in FIG. 8D, the distal end 17b of the flexible overtube 12 is stabilized within the dilated orifice 80. The flexible endoscopic shaft 22 and the endoscopic end cap 66 are then retracted from the cylindrical end cap 51 and are pulled out of the proximal end 17a (FIGS. 1, 2) of the flexible overtube 12. Once the distal end 18b of the flexible endoscopic shaft 22 is removed from within the flexible overtube 12, the endoscopic end cap 66 is removed from the distal end 18b of the flexible endoscopic shaft 22.

As shown in FIG. 8E, the flexible endoscopic shaft 22 is reinserted into the opening 24 of the flexible overtube 12. Without the endoscopic end cap 66 in place, the distal end 18b of the flexible endoscopic shaft 22 is pushed through the distal end 17b of the flexible overtube 12 through the tissue wall 80. The endoscope 14 (FIGS. 1 and 3) can now be employed to perform the intralumenal endoscopic surgical procedure at the surgical worksite.

Once the procedure is concluded, as shown in FIG. 8F, the flexible endoscopic shaft 22 is retracted through the opening 24 of the flexible overtube 12. To remove the flexible overtube 12, the distal end 17b of the flexible overtube 12 is passed through the orifice 82. The dilation balloon is inflated to dilate the orifice 82 enough to overcome the tissue gripping effect of the stability feature 48. The flexible overtube 12 is then retracted through the dilated orifice 82 and pulled out of the patient through the natural opening of the patient.

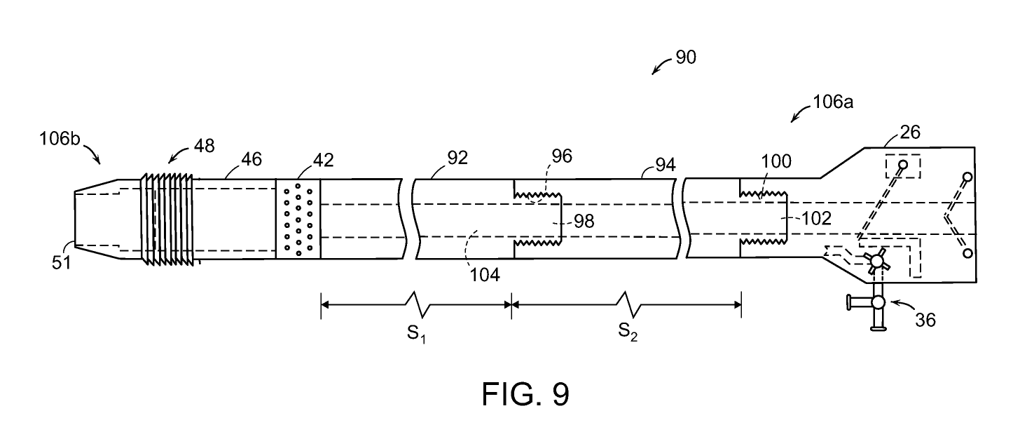

FIG. 9 illustrates one embodiment of a modular endoscopic overtube 90. The modular endoscopic overtube 90 may comprise adjustable segments at the proximal end 106a or the distal end 106b to adjust the length of the endoscopic overtube 90 or to add steerable segments at the distal end 106b. The overall length of a conventional endoscopic overtube is generally shorter than the overall length of the endoscope to enable the distal end of the flexible shaft of the endoscope to protrude though the distal end of the overtube to perform the endoscopic procedure. Longer endoscopic overtubes are easier to insert into the patient. Longer overtubes, however, interfere with the endoscopic procedure because they do not allow a sufficient length of the endoscopic flexible shaft to protrude into the body lumen or cavity (e.g., peritoneal cavity) to perform the endoscopic procedure at the worksite. The modular endoscopic overtube 90 may be employed with endoscopes having various different lengths and is easier to manufacture.

In one embodiment, the modular endoscopic overtube 90 comprises one or more modular segments such as a first removable segment 92 and a second removable segment 94. The first and second removable segments 92, 94 comprise central openings to form a central opening 104 to receive the flexible shaft of the endoscope. The distal first removable segment 92 may be a steerable segment or a straight substantially rigid segment. The removable segments 92, 94 may have variety of configurations. In one embodiment, each of the removable segments 92, 94 may have a length (e.g., S₁, S₂) from about 20 cm to about 30 cm.

The first removable segment 92 comprises a joining element 98 that is coupled to a corresponding joining element 96 of the second removable segment 94. The second removable segment 94 comprises another joining element 102 that may be coupled to another removable segment or, as shown in the illustrated embodiment, to a joining element 100 of the seal system 26. The joining elements 96, 98, 100, 102 may comprise barbs, quick connect features, or as shown in the illustrated embodiment, screw threads. The joining elements 96, 98, 100, 102 are low profile and provide a fluid tight seal and are able to be removed by the clinician during the procedure.

The removable segments 92, 94 may be removed or added before or during a procedure. Extending the length of the flexible overtube 90 by adding the removable segments 92, 84 before a procedure allows easier insertion of the flexile overtube 90 through a tissue wall inside the patient. Once the distal end 106b of the flexible overtube 90 is inserted through the tissue wall and the endoscope is advanced into the body lumen or cavity, the flexible overtube 90 may be retracted and one or more of the removable segments 92, 84 may be removed to allow some extra room for the distal end of the flexible endoscopic shaft to perform the surgical procedure at the worksite.

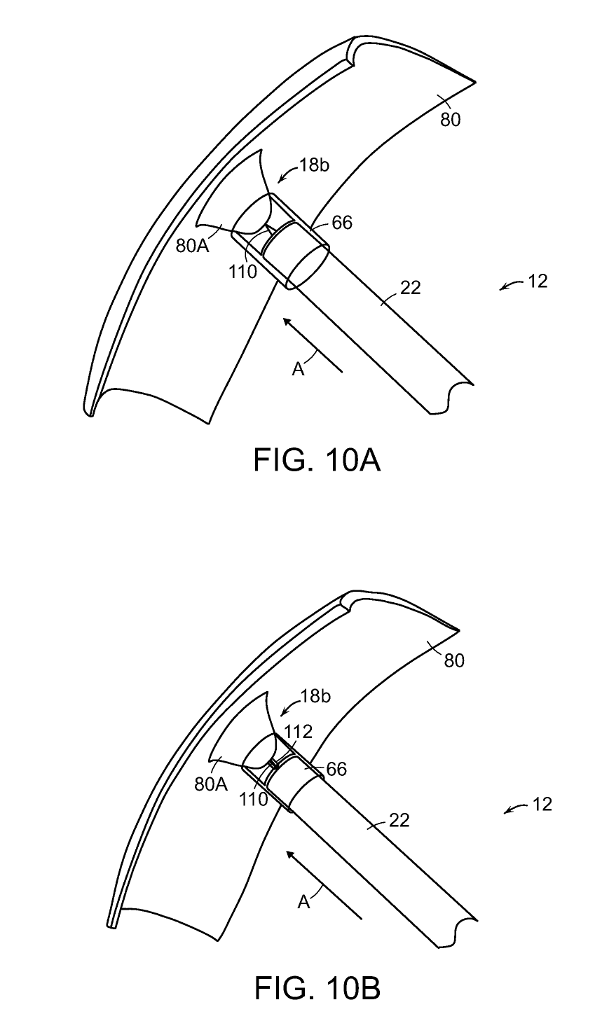

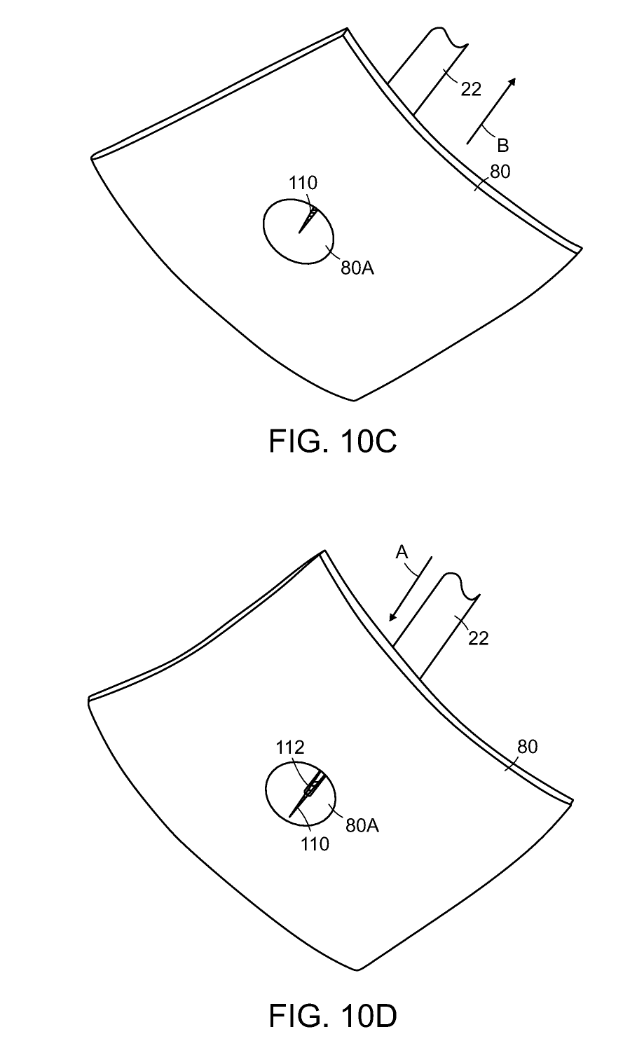

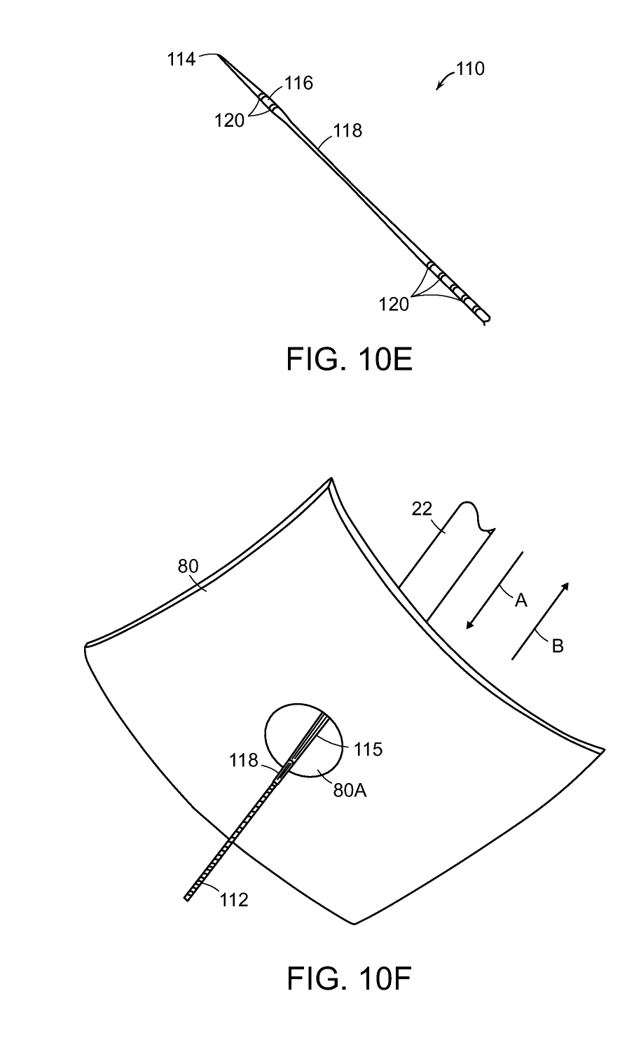

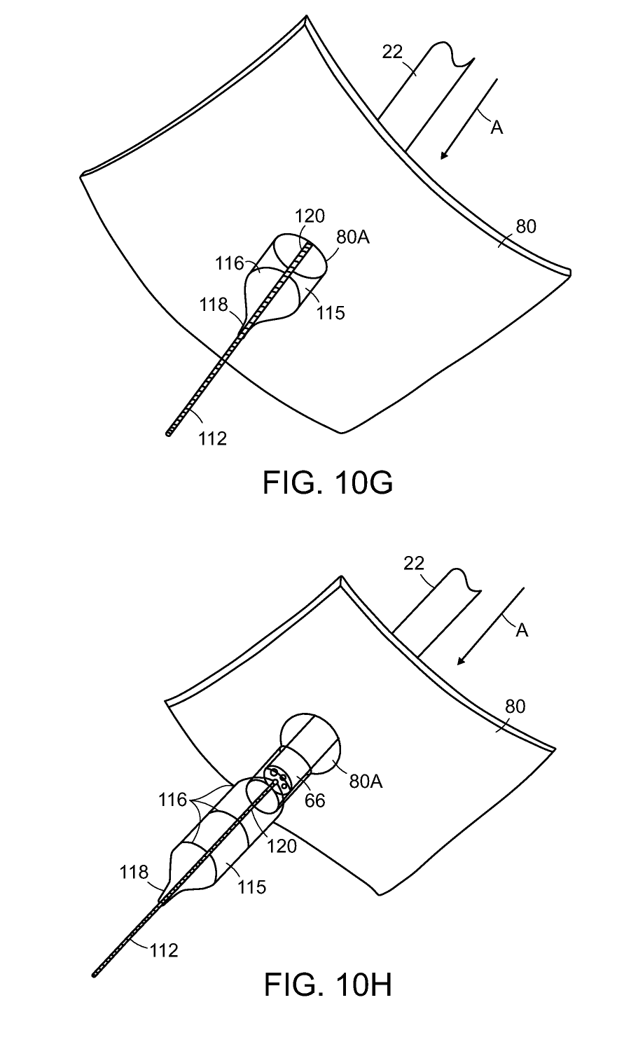

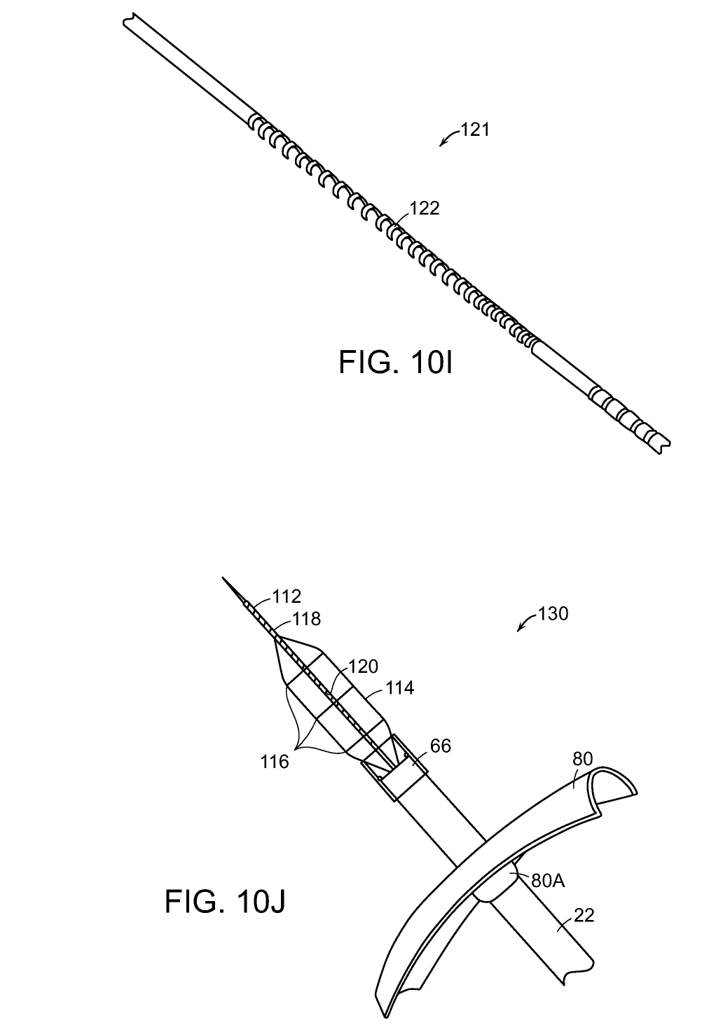

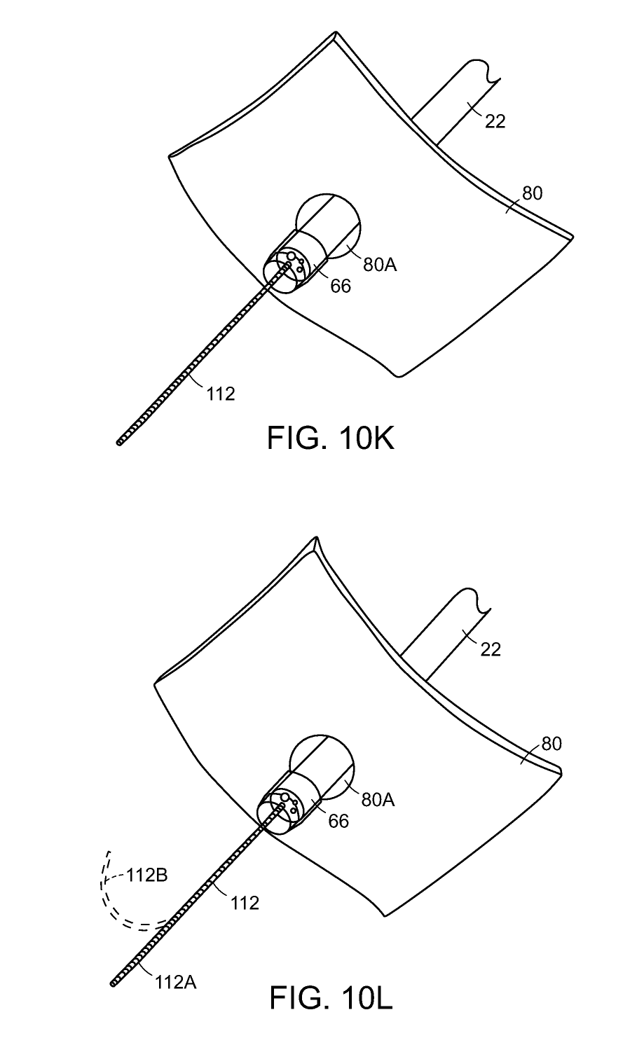

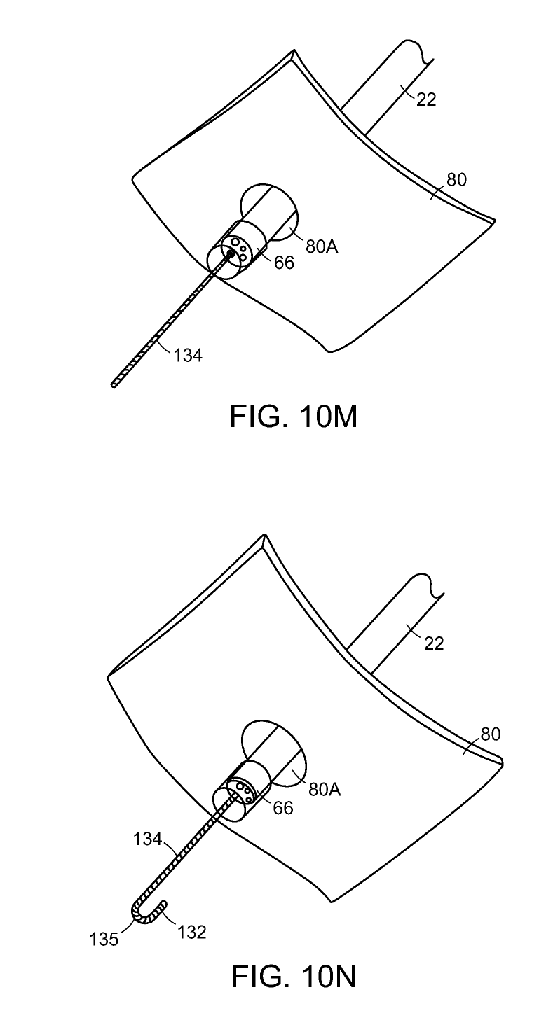

FIGS. 10A-N illustrate one embodiment of a method of introducing an endoscopic translumenal surgical device through the tissue wall of a hollow organ during an endoscopic translumenal surgical procedure. The endoscopic translumenal surgical devices should have certain attributes to minimize the severity of organ punctures when performing an endoscopic translumenal surgical procedure, especially during the initial access of the peritoneal cavity. It will be appreciated that the endoscopic translumenal surgical procedure illustrated with reference to FIGS. 10A-N may be performed using various embodiments of the flexible endoscopic translumenal overtube assembly 10 and/or the modular endoscopic overtube 90 described above. Accordingly, throughout the following description, reference also should be made to FIGS. 1-9 previously discussed.

In one embodiment of an endoscopic translumenal surgical procedure, the surgeon positions the endoscope 14 within the flexible overtube 12. The flexible endoscopic translumenal overtube assembly 10 comprising the flexible overtube 12 and the endoscope 14 are placed into a patient through a natural orifice, such as the esophagus to access the inside of a hollow organ such as the stomach. FIG. 10A, illustrates one embodiment of the flexible endoscopic shaft 22 of the endoscope inserted inside the stomach wall 80 and the distal end 18b of the endoscopic end cap 66 positioned in contact with the internal portion of the stomach wall 80. Negative pressure is applied to the endoscopic end cap 66 to isolate the portion of the tissue wall 80A to be pierced. To isolate the tissue wall 80A to be pierced, the clinician applies counter-traction. In the illustrated embodiment, this is achieved by applying suction at the distal end 18b of the flexible endoscopic shaft 22 through the endoscopic end cap 66. In other embodiments, the tissue wall 80A to be pierced may be isolated using a mechanical grabber such as a corkscrew or grasper, for example. In still other embodiments, the tissue wall 80A may be pierced without the aid of suction or other mechanical means. The endoscopic end cap 66 may have a variety of configurations. In the illustrated embodiment, the endoscopic end cap 66 is formed of a clear see-through material and enables tissue to be vacuumed or suctioned therein when a negative pressure is applied to the inside portion of the endoscopic end cap 66. The exterior surface of the endoscopic end cap 66 provides a smooth profile for tissue to glide over the entire flexible endoscopic translumenal overtube assembly 10. A solid central needle 110 is advanced in direction “A” until the solid central needle contacts the tissue wall 80A.

FIG. 10B illustrates the isolated tissue wall 80A suctioned into one embodiment of the end cap 66 and a flexible hollow tubular stylette 112 advanced over the solid central needle 110. The tubular stylette 112 may have a variety of configurations. In the illustrated embodiment, the tubular stylette 112 may be a hollow striped stylette, which slides with an internal balloon port over a striped tubular hollow stylette guidewire. In one embodiment, the tubular stylette 112 has a chamfered end. The solid central needle 110 may have a variety of configurations. In the embodiment shown in FIG. 10E, one embodiment of the solid central needle 110 comprises a sharp distal end 114 and a dilating portion 116 to pierce and spread the isolated tissue wall 80A and to minimize cutting vessels and tissue. The sharp distal end 114 and the dilating portion 116 contribute to self healing the pierced tissue wall 80A rather than bleeding out. A neck portion 118 behind the sharp distal end 114 and the dilating portion 116 enables tissue penetration only when the solid central needle 110 is sufficiently supported and guided within the tubular stylette 112. In one embodiment, the column strength of the solid central needle 110 may be reduced near the neck portion 118. Thus, if the solid central needle 110 protrudes too far outside of the tubular stylette 112, there will not be sufficient column strength to effect piercing. If the solid central needle 110 advances too far out in front of the tubular stylette 112 and a piercing force is applied to the solid central needle 110, the solid central needle 110 will bend before it pierces the tissue wall 80A. A plurality of stripes 120 are formed on the body of the solid central needle 110 to assist the clinician gage and monitor the movement and the extent of placement of the solid central needle 110 into the target tissue wall 80A site. In one embodiment, the solid central needle 110 may be an ultrasharp hollow ground needle, for example. The solid central needle 110 provides control of needle insertion speed into the isolated tissue wall 80A to be pierced because of the low insertion force of the sharp distal end 114 and the small diameter of the necked portion 118 of the solid central needle 110. This configuration requires minimal insertion force and does not create excessive potential energy storage that could cause a sudden insertion surge. Rather, the configuration provides a gradual and smooth advancement of the solid central needle 110 into the isolated tissue wall 80A to be pierced. The stripes 120 provide a visual indicator as feedback and verification to the clinician that the intended tissue wall 80A has been breached. The stripes 120 also provide feedback as to the depth of penetration of the solid central needle 110. The stripes 120 may be formed on the solid central needle 110 and/or on the tubular stylette 112. In one embodiment, this also may be achieved by providing detents on the instrument handle. In other embodiments, a tactile feedback mechanism may be provided such as a click or sudden resistance change, for example. In other embodiments, feedback may be provided by direct intramural vision during insertion using an optiview style cannula over the solid central needle 110, for example.

FIG. 10C illustrates one embodiment of the solid central needle 110 advanced to pierce or puncture the isolated stomach tissue wall 80A. It will be appreciated by those skilled in the art that the isolated stomach tissue wall 80A can be punctured without using electrocautery. The solid central needle 110 punctures the tissue wall 80A suctioned or vacuumed against the endoscopic end cap 66. Once the isolated tissue 80A is punctured, the solid central needle may be retracted in direction “B” and the tubular stylette 112 may be extended or advanced in direction A, as shown in FIG. 10D.

FIG. 10F illustrates one embodiment of the tubular stylette 112 and a deflated balloon 115 advanced through the puncture site of the isolated tissue wall 80A. The balloon 115 is positioned simultaneously in the isolated tissue wall 80A and partially inside the distal end of the flexible overtube 12 (not shown in FIG. 10F). The deflated balloon 115 is positioned behind a tapered dilating tip 118, which is ideally made of a clear see-through material. The balloon 115 is deflated when it is extended in direction A through the tissue wall 80A. The tubular stylette 112 and/or the solid central needle 110 may be left in place or may be retracted in direction B as may be needed during the procedure. In one embodiment, the balloon 115 may be formed of a clear see-through material and contain a pattern of stripes on its surface that indicate the ends of the balloon 115 and its center. For example, one thin stripe on the proximal end of the balloon 115, one thick stripe in its center, and one think stripe on the distal end of the balloon 115. Likewise, other stripe patterns may be employed.

FIG. 10G illustrates one embodiment of the balloon 115 when it is inflated to dilate the orifice formed in the tissue wall 80A at the puncture site. Enlarging or dilating the tissue wall 80A at the puncture site allows the flexible endoscopic shaft 22 to pass through the dilated opening.

FIG. 10H illustrates one embodiment of the flexible endoscopic translumenal overtube assembly 10 comprising the flexible overtube 12 and the endoscope 14 comprising the endoscopic end cap 66 advanced through the dilated opening in the tissue wall 80A. The flexible overtube 12, the endoscope 14, and the endoscopic end cap 66 are advanced after the isolated tissue wall 80A is dilated. The inflated balloon 115 exposes positioning stripes 116 to assist the clinician in placing and guiding the flexible endoscopic translumenal overtube assembly 10 through the hollow organ tissue wall 80.

As shown in FIGS. 10G, H, and I, a spring 121 may be employed to bias the tubular stylette 112 introduced over the solid central needle 110. The spring 121 may comprise a spiral kerf 122 to provide a physical shield to shroud the sharp solid central needle 110 in order to protect underlying organs from inadvertent puncture and is perceivable by the clinician. The configuration wherein the spring 121 is used to shroud the sharp solid central needle 110 may be referred to as a veress needle configuration. Those skilled in the art will appreciate that a veress needle is a needle equipped with a spring loaded obturator that is used for insufflation of the abdomen in laparoscopic or endoscopic surgeries. In other embodiments, means may be provided to automatically terminate the piercing function after the isolated tissue wall 80A has been pierced or breached.

FIG. 10J is an overall view of the endoscopic translumenal surgical system 130 described above.

As shown in FIG. 10K, the balloon 115 has been deflated and withdrawn. This leaves the tubular stylette 112 and/or the solid central needle 110 behind to be used as a guidewire.

As shown in FIG. 10L, the tubular stylette 112 has a flexible feature and can be articulated from a straight position 112A to a flexed position 112B, shown in broken line, by the clinician.

FIG. 10M illustrates one embodiment of a flexible central needle 134. In the illustrated embodiment, the flexible central needle 134 is formed with a smaller neck portion at the distal end to allow the solid central needle 134 to flex. When pulled mostly inside the balloon 115 catheter and the tubular stylette 112, the column strength of the solid central needle 110 is very strong, and therefore will puncture the tissue wall 80 suitably well.

As shown in FIG. 10N, the flexible central needle 134 is fully extended (unsupported) and is shown in the flexed state with a flexed portion 132. In the flexed state, the flexible central needle 134 presents a blunt distal 135 end and will not puncture tissue. Thus, the flexible central needle 134 can be flexed and used as guidewire, for example. In one embodiment, the flexible central needle 134 may be drawn inside the tubular stylette 112 and housed therein during use as a guidewire.

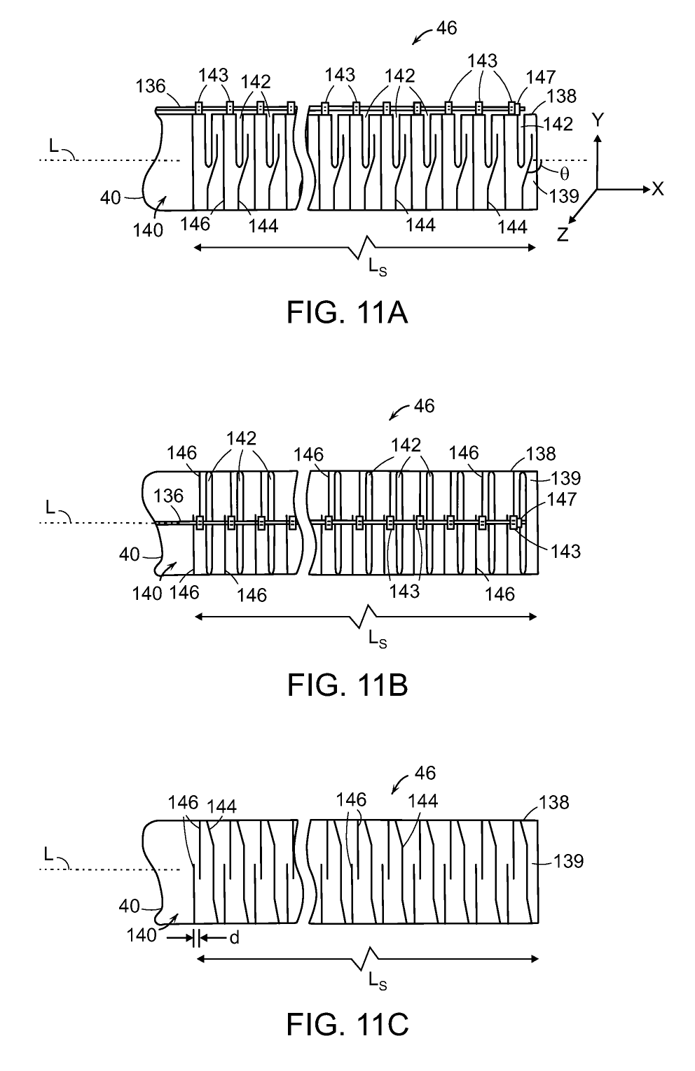

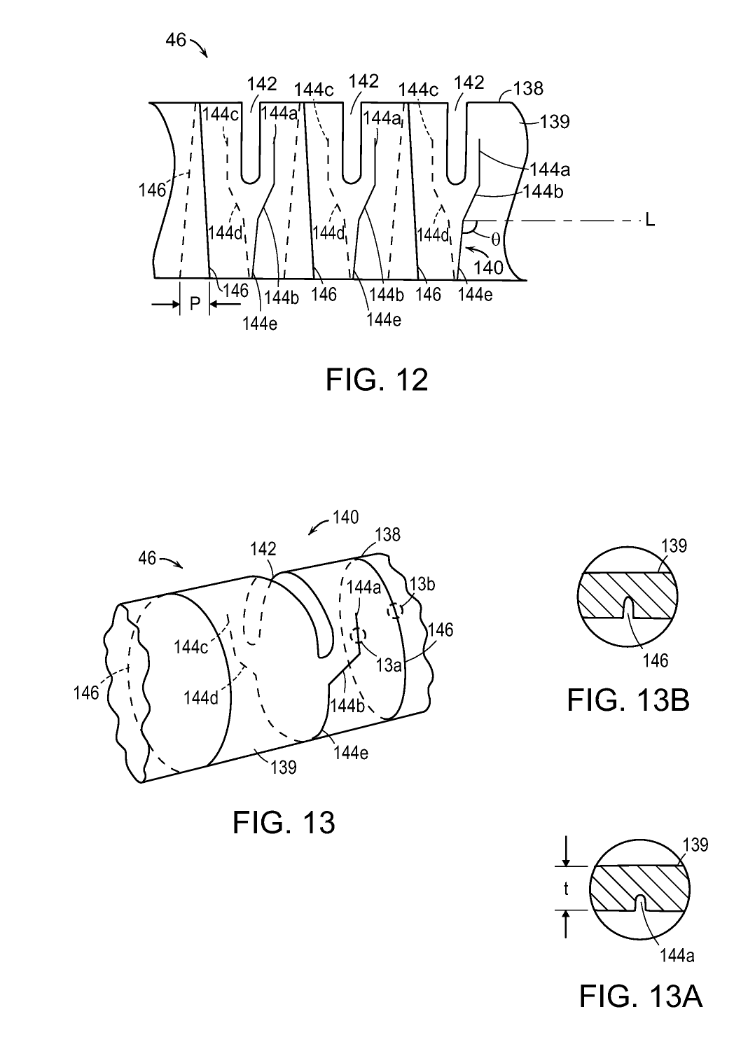

FIGS. 11A-C illustrate one embodiment of an actively articulatable steerable tube 138 portion of the steerable segment 46. The steerable tube 138 is shown without a protective layer that is slidably received over the steerable tube 138 to form a fluid tight seal. FIG. 12 is a side view of one embodiment of the steerable segment shown in FIGS. 1A-C. FIG. 13 is a perspective view of a portion of the steerable segment shown in FIGS. 11A-C and 12. FIG. 13A is a cross-sectional view of a wall portion of the steerable segment shown in FIG. 13. FIG. 13B is a cross-sectional view of a wall portion of the steerable segment shown in FIG. 13.

In the embodiment illustrated in FIGS. 11A-C, the steerable tube 138 comprises a series of slits 140 cut into the body 139 defining a pattern of articulatable elements to enable active articulation of the steerable tube 138 in a first plane XY and passive deflection in planes XZ and YZ that are orthogonal to the first plane. The slits 140 may be cut into the steerable tube 138 in a variety of patterns to assist with flexure in the direction of the pull cable 136 plane (XY in the embodiment illustrated in FIG. 11A). In one embodiment, the steerable segment 46 and/or the steerable tube 138 has a length L_{S} of about 20 cm.

In one embodiment, the steerable tube 138 comprises a pattern of slits 140 cut in a pattern on the body 139. In one embodiment, the pattern of slits 140 comprises a series of apertures 142, S-shaped slits 144, and spiral slits 146 formed along the longitudinal length of the body 139 of the steerable tube 138. As shown in FIGS. 11A-C, the pattern of slits 140 is repeated along the longitudinal axis L. In one embodiment, the apertures 142 are about 1 mm wide and spaced apart by about 4 mm. The S-shaped slits 144 begin on one side of the body 139 of the steerable tube 138 and wrap around to the other side. The S-shaped slits 144 comprise a first portion 144a that is perpendicular to the longitudinal axis “L” of bending. The first portion 144a has a length of about 6 mm. A second portion 144b forms an angle θ between about 100 to about 110 degrees with the longitudinal axis “L” and has a length of about 6 mm (FIGS. 11A and 12). A third portion 144c is parallel to the first portion 144a and has a length of about 10 mm. A fourth portion 144d is parallel to the second portion 144b and has a length of about 6 mm. A fifth portion 144e is parallel to the first portion 144a and the third portion 144c and has a length of about 6 mm. The spiral slits 146 are in the form of a helix and make one revolution around the body 139 of the steerable tube 138 with an overlap “d” (FIG. 11C) of about 1.5 mm and a pitch “p” of about 1.25 mm. In the illustrated embodiment, the spiral slits 146 are positioned between the apertures 142 and the S-shaped slits 144. In other embodiments, the apertures 142, S-shaped slits 144, and spiral slits 146 may be positioned relative to each other in any predetermined arrangement.

The steerable tube 138 is attached to at least one of the pull cables 136 such that it can be actively articulated in the XY plane away from the neutral longitudinal axis “L.” In one embodiment, in the active articulation direction, e.g., the XY plane, the steerable tube 138 can be articulated through angles up to about 180 degrees when tension is applied to the pull cable 136 and can passively flex about 45 degrees in the directions orthogonal to the active articulation direction. In another embodiment, the steerable tube 138 can be passively articulated in the XZ plane orthogonal to the XY plane defined by the pull cable 136 through angles up to about 90 degrees. The pull cable 136 may be loosely threaded through a series of rings 143 disposed along an outer portion of the hollow body 139 along the longitudinal length of the steerable tube 138 and is fixedly attached to at least one of the rings 143 such that the steerable tube 138 bends when tension is applied to the pull cable 136. In the illustrated embodiment, the distal end of the pull cable 136 is fixedly attached to the ring 143 located at the distal end of the steerable tube 138 by a crimp, lock, or knot feature 147 to prevent the pull cable 136 from being pulled through the distal ring 143. Thus, when tension is applied to the pull cable 136, the flexible tube 138 bends or articulates in the XY plane defined by the pull cable 136.

To assist straightening of the steerable segment 46, the body 139 of the steerable tube 138 may be constructed of full-hardened steel that tends to spring back more readily than softened annealed metal. In another embodiment, a straightening member may be disposed along the longitudinal axis “L” to provide a spring force that tends to straighten the steerable segment 46 when tension in the pull cable 136 is released. The straightening member may be made from a superelastic alloy such as NITINOL® wire, spring steel, music wire, or other material having a suitable level of elastic deformation and stored energy to straighten the steerable segment 46. In one embodiment, the straightening member may be positioned adjacent to the pull cable 136 and may be allowed to freely float on its proximal end. In another embodiment, the straightening member may be positioned orthogonal to the pull cable 136 and periodically fixed to the surface of the steerable tube 138 through welds or other connection means.

Referring now to FIGS. 1, 2, 4, and 11-13B, in one embodiment, the steerable segment 46 may comprise the steerable tube 138 shown in FIGS. 11A-C, 12, 13A, 13B (FIGS. 11-13B). As shown in FIGS. 1 and 2, the steerable segment 46 is located at the distal end 17b of the flexible overtube 40. The steerable tube 138 comprises an elongate hollow body 139 defining a central opening suitable for receiving an endoscope therein. A series of slits 140 are formed into the body 139 defining a plurality of articulatable elements to make the steerable tube 138 flexible while still providing sufficient column strength to advance the steerable tube 138 through a passageway leading to a body cavity within the patient. In one embodiment, the inside diameter of the steerable tube 138 may be selected to enable an endoscope to freely slidably move within the steerable tube 138 when it is articulated. For example, the inside diameter of the steerable tube 138 may be about 10 mm for a single channel diagnostic endoscope and about 15 mm for a two-channel endoscope.

The steerable tube 138 may be formed of a variety of materials including metallic materials, steel, brass, polycarbonate, polyetheretherketone (PEEK), urethane, or polyvinylchloride (PVC). In one embodiment, the steerable tube 138 may be constructed of full-hardened steel that tends to spring back more readily than softened annealed metal. The wall thickness “t” of the body 139 of the steerable tube 138 may range from about 0.25 mm to about 1 mm.

In one embodiment, the series of slits 140 may be formed with a laser cutter. In other embodiments, the series of slits 140 may be formed with a machine bit or other suitable means for forming a substantially narrow cut, opening, or aperture, for example. In one embodiment, the series of slits 140 may be cut into the body 139 in a predetermined pattern without removing sections or portions of the material other than the kerf. As shown in FIGS. 13A and 13B portions of the series of slits 140, such as the S-shaped slits 144 and the spiral slits 146, for example, may be formed on an outer surface portion of the body 139 without entirely penetrating the wall thickness “t” of the body 139. In another embodiment, the series of slits 140 may be formed by removing sections or portions of the material along its length. In yet another embodiment, the series of slits 140 may be formed by creating a mold of a desired form and shape and then molding the steerable tube 138 using conventional plastic molding techniques. It will be appreciated that any combination of these techniques may be employed to form the series of slits 140 in a predetermined pattern defining a plurality of articulatable elements that render the steerable tube 138 flexible yet sufficiently rigid to provide adequate column strength for insertion through a passageway leading to a body cavity within the patient. The embodiments are not limited in this context.

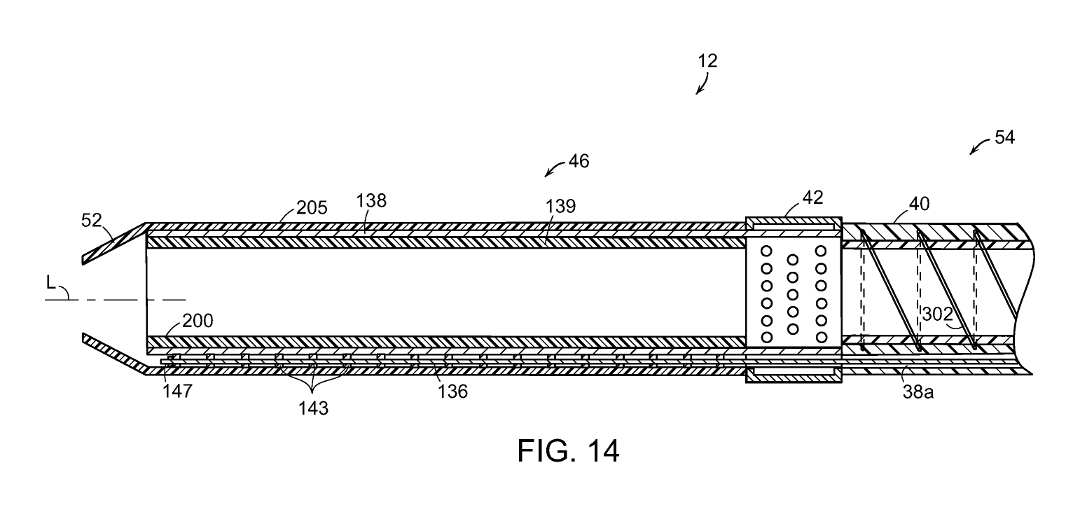

FIG. 14 is a cross-sectional view of the steerable segment 46 and the middle segment 54 of the flexible overtube shown 12 shown in FIGS. 1 and 2. In one embodiment, the steerable segment 46 may comprise the steerable tube 138, previously described with reference to FIGS. 11-13B, comprising a first layer of flexible material disposed on an inner portion of the steerable tube 138 and a second layer of flexible material disposed on an outer portion of the steerable tube 138 to maintain a fluid tight seal. In one embodiment, the steerable segment 46 comprises an inner woven boot 200, an outer flexible boot 205, and the steerable tube 138 coaxially floating between the inner woven boot 200 and the outer flexible boot 205. The inner woven boot 200, the outer flexible boot 205, and the steerable tube 138 are connected at their proximal and distal ends. The inner woven boot 200 can be constructed of polypropylene, or polyethylene strands woven into a tube having an inner diameter of about 15.5 mm. The outer flexible boot 205 may be extruded or molded from one continuous piece. In one embodiment, the tapered segment 52 and stability threads (not shown in FIG. 14 for clarity) may be formed integrally with the molded outer flexible boot 205 component. The outer flexible boot 205 is placed over the steerable tube 138 and is fixed at the distal and proximal ends through heat forming, epoxy or other adhesives. Suitable materials for the outer flexible boot 205 include polyurethane, isoprene, fluoroelastomer (VITON®), silicone, or other flexible materials. In one embodiment, the middle segment 54 comprises a flexible polymeric tube, e.g., the flexible sheath 40, reinforced by an embedded spring 302. The spring 302 has a wire diameter of about 0.310 mm and the outer coil diameter of the spring 302 may range from about 7 mm to about 17 mm. The spring 302 may be sandwiched between two layers of polymer, such as polyurethane, silicone, polymers (PEBAX®), or other suitable material. The outer diameter of the middle segment 54 may range from about 8 mm to about 18 mm.

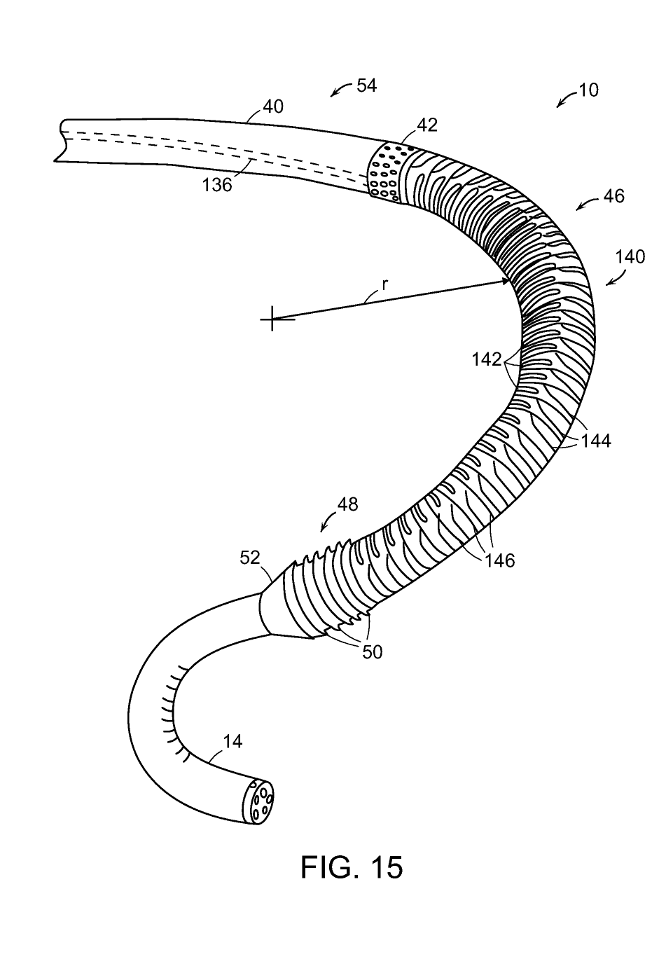

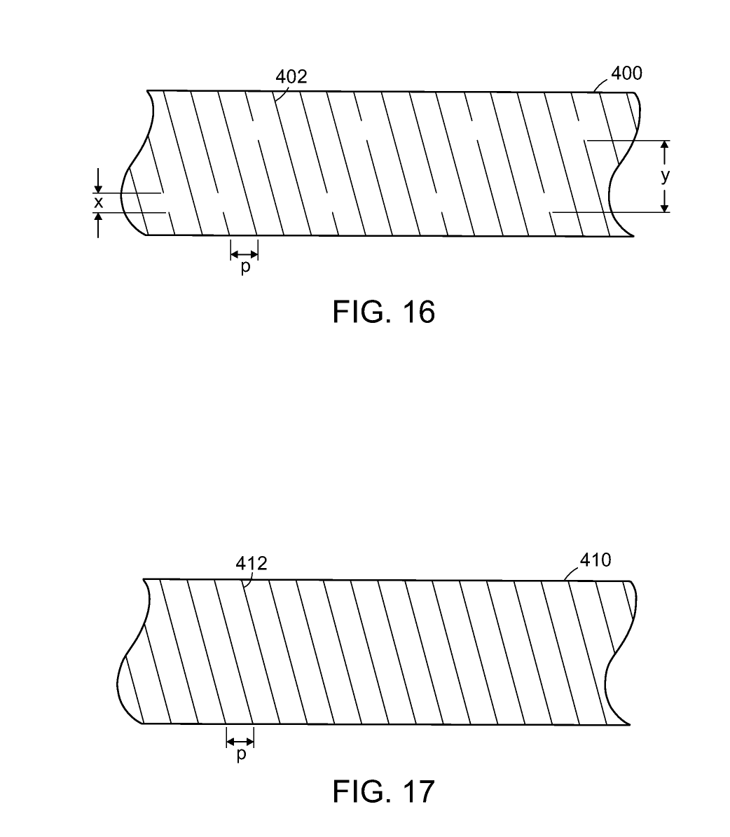

FIG. 15 illustrates the steerable segment 46 of the flexible endoscopic translumenal overtube assembly 10 shown in FIG. 1 in an actuated state. With reference now to FIGS. 1, 2, and 11-15, the pull cable 136 extends from a distal portion of steerable segment 46, through the first lumen 38a, and extends out of the proximal end of the flexible sheath 40. As previously discussed, the distal end of the pull cable 136 is fixedly attached to the distal end of the steerable tube 138 by a crimp, lock, or knot feature 147 to prevent the pull cable 136 from being pulled through a distal ring 143. Thus, when tension is applied to the pull cable 136 by the actuation handle 300, the steerable segment 46 bends in the XY plane away from its neutral axis in a radius of curvature “r” through angles up to about 180 degrees as may be defined by the pattern or series of slits 140 in the active articulation direction and about 45 degrees of passive flexion in the directions orthogonal to the active direction. A desired radius of curvature “r” allows the endoscope 14 to be inserted and withdrawn without the need to straighten the steerable segment 46. A suitable bend radius is between about 3 cm to about 5 cm. The portion extending beyond the flexible sheath 40 is contained in the coil pipe assembly 210 that extends to the actuation handle 300.

FIG. 16 is a side view of one embodiment of a steerable overtube comprising an interrupted spiral cut pattern of slits. In one embodiment, the steerable segment 46 may comprise a steerable overtube 400 comprising slits 402 formed is an interrupted spiral cut pattern as shown in the embodiment illustrated in FIG. 16. The interrupted spiral cut pattern slits 402 may be defined by pitch “p,” depth of interruption “x,” and distance between interruptions “y.” Each of these variables may be varied to achieve a desired bending radius, flexibility, and/or torquability of the steerable segment 400. In one embodiment, the interrupted spiral cut pattern slits 402 may be defined by a pitch “p” of about 1.5 mm, a depth of interruption “x” of about 16 mm, and a distance between interruptions “y” of about 1.5 mm. The interrupted spiral cut pattern slits 402 may be formed using a laser cutter. The width of the interrupted spiral cut pattern slits 402 is limited to the width of the cutting element, e.g., the laser spot site, which may be less than about 0.0254 mm.

FIG. 17 is a side view of one embodiment of a steerable overtube comprising a spiral cut pattern of slits. In one embodiment, a steerable overtube 410 comprises slits 412 formed in a spiral cut pattern. The spiral cut pattern slits 412 may be defined by a pitch “p,” which may be varied to achieve a desired bending radius, flexibility, and/or torquability of the steerable segment 410. In one embodiment, the spiral cut pattern slits 412 may be defined by a pitch “p” of about 1.5 mm. The spiral cut pattern slits 412 may be formed using a laser cutter or machine bit. The width of the spiral cut pattern slits 412 may be less than about 0.0254 mm, and is limited by width of the laser spot site.

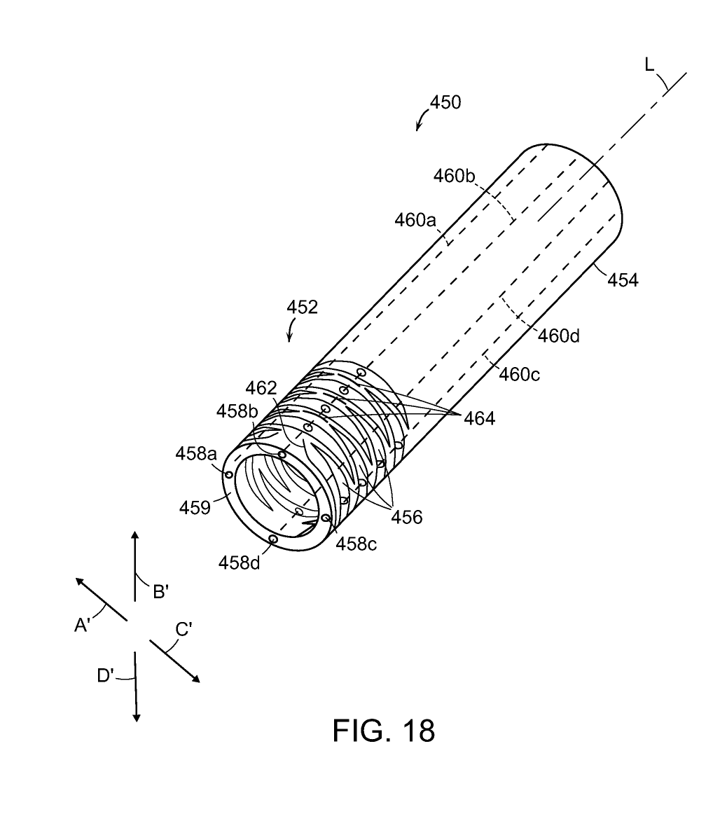

FIG. 18 illustrates one embodiment of a steerable segment 450 comprising a steerable overtube 452 and a flexible segment 454. The flexible segment 454 may be substantially similar to the flexible overtube 12 previously described. The steerable overtube 456 comprises an elongate hollow body 459 defining a central opening suitable for receiving an endoscope therein. A plurality of apertures 456 and slits 462, 464 are formed on the body 459 of the steerable overtube 452 to make it flexible while still providing sufficient column strength. In one embodiment, the apertures 456 may be substantially similar to the apertures 142; the slits 462 may be substantially similar to the S-shaped slits 144; and the slits 464 may be substantially similar to the spiral slits 146; all of which are previously described with reference to FIGS. 11-13B. A plurality of embedded lumens 458a, 458b, 458c, and 458d are formed in the body 459 and extend along the longitudinally along axis “L.” The lumen 458a-d also extend along the flexible segment 454. The embedded lumens 458a-d may have a diameter of about 1 mm. A plurality of pull cables 460a, 460b, 460c, 460d are disposed within the corresponding lumens 458a-d. The pull cables 460a-d are coupled to an actuation handle (not shown) at a proximal end. The actuation handle may be configured to apply tension to any one of or any combination of the pull cables 460a-d to articulate the steerable overtube 452 in any corresponding direction indicated by arrows A′, B′, C′, and D′. The steerable overtube 452 may be articulated in other directions by applying tension to a combination of pull cables 460a-d.

As indicated above, the various devices disclosed herein can be used in a variety of surgical procedures, including endoscopic procedures, laparoscopic procedures, and in conventional open surgical procedures, including robotic-assisted surgery. In one exemplary endoscopic procedure, an elongate shaft of a surgical device, such as one previously disclosed herein, can be inserted through a natural orifice and a body lumen to position an end effector located at a distal end of the elongate shaft adjacent to tissue to be treated.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the embodiments described herein will be processed before surgery. First, a new or used instrument is obtained and if necessary cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that device is sterilized. This can be done by any number of ways known to those skilled in the art including beta or gamma radiation, ethylene oxide, steam. One skilled in the art will appreciate further features and advantages of the above-described embodiments. Accordingly, the embodiments are not to be limited by what has been particularly shown and described, except as indicated by the appended claims.

1. An apparatus for use with an endoscope, the apparatus comprising:
a flexible overtube comprising a proximal end and a distal end defining a hollow lumen therebetween to receive a flexible shaft portion of an endoscope therein, the proximal end of the flexible overtube is configured to remain outside of a patient and the distal end is configured to enter the patient through a natural orifice; and
at least one fluid tight seal located at the proximal end to prevent leakage of fluids around the flexible shaft of the endoscope when the flexible shaft of the endoscope is positioned within the flexible overtube.

2. The apparatus of claim 1, comprising:
an additional fluid tight seal located near the proximal end of the flexible overtube to prevent leakage of fluid through the hollow lumen defined within the flexible overtube when no other devices are positioned within the hollow lumen.

3. The apparatus of claim 1, wherein the at least one fluid tight seal provides a seal around the flexible shaft of the endoscope having a size in the range of between about 5 mm to about 13 mm.

4. The apparatus of claim 1, comprising:
an opening distal to the at least one fluid tight seal, wherein the opening can be selectively opened and closed to allow passage of fluids from inside the hollow lumen of the flexible overtube to the outside of the flexible overtube.

5. The apparatus of claim 4, wherein the opening comprises means for connecting to a fluid source.

6. The apparatus of claim 5, wherein the connection comprises a female luer connection.

7. The apparatus of claim 1, wherein the flexible overtube is disposable after a single use.

8. The apparatus of claim 1, comprising a stability feature located at the distal end of the flexible overtube to position the distal end of the flexible tube within the patient's body.

9. The apparatus of claim 8, wherein the stability feature comprises a plurality of annular rings on the outer surface of the flexible overtube having a triangular cross section.

10. The apparatus of claim 8, wherein the stability feature comprises a balloon on the outer surface of the flexible overtube to allow easy passage through a tissue wall when deflated and to maintain the position of the distal end when inflated.

11. The apparatus of claim 1, comprising at least one fluid tight lumen embedded within a wall of the flexible overtube that extends along the longitudinal length of the flexible overtube, the at least one fluid tight lumen comprising a proximal end and a distal end.

12. The apparatus of claim 11, wherein the at least one fluid tight embedded lumen has an inner diameter of about 1 mm.

13. The apparatus of claim 11, comprising a collar on the outside surface of the flexible overtube that is connected to the distal end of the at least one fluid tight lumen, wherein the collar is configured to draw fluid from the inside of a patient's body into the at least one fluid tight lumen when the proximal end of the at least one fluid tight lumen is connected to a negative pressure source.

14. The apparatus of claim 11, comprising:
a pull cable disposed within the at least one fluid tight embedded lumen; and
a steerable segment located at the distal end of the flexible overtube that can be actively articulated away from a neutral axis through angles up to about 180 degrees when tension is applied to the pull cable.

15. The apparatus of claim 14, wherein the steerable segment comprises:
a metal tube comprising a series of slits defining a pattern to enable active articulation in a first plane and passive deflection in planes orthogonal to the first plane.

16. A flexible endoscopic translumenal overtube system, comprising:
a flexible endoscope comprising a flexible shaft; and
a flexible overtube comprising a proximal end and a distal end defining a hollow lumen therebetween to receive the flexible shaft of an endoscope therein, the proximal end of the flexible overtube is configured to remain outside of a patient and the distal end is configured to enter the patient through a natural orifice; and
at least one fluid tight seal located at the proximal end to prevent leakage of fluids around the flexible shaft of the endoscope when the flexible shaft of the endoscope is positioned within the flexible overtube.

17. The flexible endoscopic translumenal overtube system of claim 16, comprising:
an additional fluid tight seal located near the proximal end of the flexible overtube to prevent leakage of fluid through the hollow lumen defined within the flexible overtube when no other devices are positioned within the hollow lumen.

18. The flexible endoscopic translumenal overtube system of claim 16, wherein the at least one fluid tight seal provides a seal around the flexible shaft of the endoscope having a size in the range of between about 5 mm to about 13 mm.

19. The flexible endoscopic translumenal overtube system of claim 16, comprising:
at least one fluid tight lumen embedded within a wall of the flexible overtube that extends along the longitudinal length of the flexible overtube, the at least one fluid tight lumen comprising a proximal end and a distal end.

20. A method of using a flexible endoscopic translumenal overtube system, the method comprising:
positioning a flexible shaft of an endoscope within a hollow lumen defined by a flexible overtube;
placing the flexible overtube and the flexible shaft of the endoscope into a patient through a natural orifice of the patient;
puncturing an organ wall using a needle advanced through a working channel of the endoscope;
dilating the puncture site and forming an opening by inflating a balloon positioned simultaneously in the tissue and partially inside the distal end of the flexible overtube; and
advancing the flexible overtube and the endoscope through the dilated opening in the organ wall of the patient.

21. A flexible trocar apparatus for use with an endoscope, the apparatus comprising:
a flexible overtube comprising a proximal end and a distal end defining a hollow lumen therebetween to receive a flexible shaft portion of an endoscope therein, the proximal end of the flexible overtube is configured to remain outside of a patient and the distal end is configured to enter the patient through a natural orifice;
at least one fluid tight seal located at the proximal end to prevent leakage of fluids around the flexible shaft of the endoscope when the flexible shaft of the endoscope is positioned within the flexible overtube;
a segment that is passively flexible; and
a distal portion that is actively steerable in one plane and passively flexible in the orthogonal planes.

22. The flexible trocar apparatus of claim 21 wherein the actively steerable section bends to and is held in a radius of curvature suitable to slidably receive an endoscope therethrough.

23. A flexible trocar apparatus for use with an endoscope, the apparatus comprising:
a trocar housing at a proximal end comprising at least one fluid tight seal located at the proximal end to prevent leakage of fluids around the flexible shaft of the endoscope when the flexible shaft of the endoscope is positioned within the flexible overtube;
a passively flexible overtube portion in a middle portion comprising a proximal end and a distal end defining a hollow lumen therebetween to receive the flexible shaft of an endoscope therein; and
an actively articulatable portion at a distal end.

24. The apparatus of claim 23 further comprising:
a suction collar that fluidically communicates to the exterior surface of the apparatus and is fluidically separate from the interior surface of the apparatus.

25. The apparatus of claim 23, wherein the actively articulatable portion comprises a metal tube slit with a pattern that allows 180 degrees bend in the active articulation direction and 45 degrees passive flexion in the directions orthogonal to the active direction.

26. The apparatus of claim 23, wherein the passively flexible portion further comprises at least one additional smaller lumen that is fluidically separate from the larger hollow lumen
